(19) United States
(12) Patent Application Publication
(10) Pub. No.: US 2010/0010627 A1
(43) Pub. Date: Jan. 14, 2010

(54) Breast Implants and Compositions of Extracellular Matrix

(76) Inventor: Robert G. Matheny, Norcross, GA (US)

Correspondence Address: Ballard Spahr LLP, SUITE 1000, 999 PEACHTREE STREET, ATLANTA, GA, 30309-3915, US

(21) Appl. No.: 12/428,766

(22) Filed: Apr. 23, 2009

Related U.S. Application Data
(63) Continuation of application No. 11/746,981, filed on May 10, 2007, now abandoned.

(51) Int. Cl. A61F 2/12

(52) U.S. Cl. 623/8

(57) ABSTRACT

The invention is articles and compositions of extracellular matrix for forming breast implants and otherwise augmenting or reconstructing breast tissue and other cosmetically desired tissue in humans, such as lips and cheeks. The invention is also to methods of using these in implant articles to augment or reconstruct a human breast or other tissue.

### CROSS-REFERENCE TO RELATED APPLICATIONS

This application is a continuation of copending application Ser. No. 11/746,981, filed May 10, 2007, which is hereby incorporated herein by reference in its entirety.

### FIELD OF THE INVENTION

The invention relates to augmenting or reconstructing a human breast using novel articles and compositions of mammalian extracellular matrix material.

### BACKGROUND OF THE INVENTION

Tissue regeneration has been accomplished by using extracellular matrix material derived from mammalian tissues. Some of these mammalian tissues that have been described in patent literature include small intestine submucosa (SIS), liver basement membrane (LBM), urinary bladder submucosa (UBS) and stomach submucosa (SS). See U.S. Pat. No. 5,554,389, U.S. Pat. No. 4,902,508, and U.S. Pat. No. 5,281,422. Enamel matrices, which are the extracellular matrix around forming teeth, are described in U.S. Pat. No. 7,033,611. Extracellular matrices from these tissues have been isolated and dried to become solid materials (sheets and particulates). Particulate forms can be rehydrated in a suitable buffer to become fluidized or emulsive forms. Presently, these extracellular matrix compositions are used for tissue grafting, wound healing, and tissue regenerative purposes.

### SUMMARY OF THE INVENTION

The invention is an article for placing in a human breast comprising one or more sheets of mammalian extracellular matrix encasing a composition comprising extracellular matrix in an emulsion, gel, paste, liquid or particulate form, wherein the article forms an implant shaped to conform to a shape of a human breast.

The invention is also a method of augmenting or reconstructing a human breast comprising providing an article comprising one or more sheets of mammalian extracellular matrix encasing a composition comprising extracellular matrix in an emulsion, gel, paste, liquid or particulate form, wherein said article forms an implant shaped to conform to a shape of a human breast, and placing said article in a human breast.

The invention is further a method of augmenting tissue at a site in the human body comprising: providing a composition comprising mammalian extracellular matrix, and introducing said composition into said human body in sufficient amount to generate new tissue at said site, resulting in an augmentation of tissue at said site. The composition can comprise extracellular matrix from more than one mammalian tissue source. The composition can comprise extracellular matrix in a concentration greater than about 0.001 mg/ml. The site in said human body can be a breast.

### BRIEF DESCRIPTION OF THE DRAWINGS

FIG. 1 depicts a cross sectional views of a breast implant of extracellular matrix sheets encasing a gel of extracellular matrix.

FIG. 2 depicts a view looking down on the breast implant article.

FIG. 3 depicts a cross sectional view of the breast implant in a human breast.

FIG. 4 depicts a cross sectional view of introduction of extracellular matrix material into a human breast for augmentation purposes.

FIG. 5 depicts sectional views of introduction of extracellular matrix material into human lips for augmentation purposes.

FIG. 6 depicts sectional views of introduction of extracellular matrix material into human cheeks for augmentation purposes.

### DETAILED DESCRIPTION OF THE INVENTION

The invention is an article made of extracellular matrix sheets sandwiching a composition of extracellular matrix emulsion, gel, liquid, paste or particulate. The article is useful for placing in a mammal in need of tissue regeneration or tissue augmentation in order to effect tissue regeneration or augmentation at the site of placement of the article. The extracellular matrices used in the article can be from one or more than one source of extracellular matrix in a mammal. The article can also be made of extracellular matrix components (i.e. sheets and gels) that are advantageously derived from different sources of extracellular matrix in one or more donor mammals. A composition of extracellular matrix can also be injected into a particular site of the body in order to achieve tissue augmentation, such as for example, the breast, lips, cheeks, or buttocks of a patient.

The article of the invention is made up of a composition comprising an emulsion, gel, paste, liquid or dry particulate of extracellular matrix that is encased in one or two sheets of extracellular matrix. The encasing of the emulsion or particulate can be accomplished by laminating the ends of the sheets to enclose the composition inside. The composition can be sandwiched between two sheets of extracellular matrix with the edges of the sheets laminated together, or somehow made to close either fully or partially to encase the composition inside. For an article for augmenting a human breast, the sheets encasing the composition will be contoured to conform to the shape of a human breast.

The sheets can be from the same source of extracellular matrix, i.e. both or all sheets can be made of SIS from a pig. The sheets can also be from different sources of extracellular matrix, for example the first sheet is SIS, and the second sheet is SS. Both the SIS and SS can be from the same species of mammal (e.g. pig) or each from a different species of mammal (e.g. SIS from pig, and SS from cow).

he article can be made up of sheets of extracellular matrix from one tissue source and a composition comprising extracellular matrix from another, different tissue source. So, for example, the sheets can be SIS and the emulsion, gel, paste, liquid or particulate composition can be LBM; or the sheets can be SIS and the emulsion, gel, paste, liquid or particulate composition can also be SIS. All these combinations are exemplary and not meant to represent limitations in the possible combinations of forms and sources of extracellular matrix for forming the articles of the invention.

The sheets can be laminated to each other at the edges around an amount of composition comprising the emulsion, gel or particulate extracellular matrix that then becomes encased in the two sheets upon lamination of the outer sheets to each other. The lamination of the two outer sheets together can be partial or complete, so that the composition inside can be entirely contained within the two sheets, or can be permitted to ooze out from between the sheets upon placement in the subject receiving treatment. The two sheets may also be attached to each other by quilting of the sheets in the middle of the sheets much like a quilt is assembled when made of 2 or more layers of fabric. In order to form an article that is shaped like a human breast, several sheets may be laminated together to form the breast-like shape. The base of the article can be flat which conforms to the base of the human breast, and the top of the article will need to rise in a mound to conform to the natural or desired shape of the breast being augmented. The gel or emulsion of extracellular matrix that is encased in the article will aide in supporting the shape of the shell that is provided by the sheets of extracellular matrix and should substantially fill the shell shaped by the sheets of extracellular matrix.

In one embodiment, the two sheets of extracellular matrix can encase or retain a composition that comprises particulate matrix. So that two sheets can surround or encase a composition comprising an amount of dry particulate extracellular matrix and the sheets can be laminated to each other around the particulate and therefore encase it. The particulate can be of the same or different source of extracellular matrix as the sheets. So that for example, the sheets can be made from SIS and the particulate can be from LBM.

The emulsion, gel, paste, liquid or particulate placed in between the sheets of matrix can be of mixed source of extracellular matrix, so that for example an emulsion, gel, paste, liquid or particulate can be a 50:50 mixture of LBM and UBS or essentially any ratio ranging from 1:99 to 99:1. Thus, the emulsion, gel, paste, liquid or particulate that comprises the composition placed between two sheets of extracellular matrix can be some mixture or ratio of extracellular matrix from one or more tissue sources, or it can be from a single tissue source.

Mammalian tissue sources are in general any tissue having an extracellular matrix that can be isolated from a mammal and de-cellularized. Thus for example, most mammalian organs are tissue sources. The tissue sources can be for example any mammalian tissue, including but not limited to the small intestine, large intestine, stomach, lung, liver, kidney, pancreas, placenta, heart, bladder, prostate, tissue surrounding growing tooth enamel, tissue surrounding growing bone, and any fetal tissue from any mammalian organ.

The forms of the extracellular matrices that make up the articles are generally any form of extracellular matrix, including forms such as sheets, or other forms that result from human manipulation with the extracellular matrix. Generally the broad categories of forms appear to be liquid, semi-solid, or solid. Liquid is generally a thin emulsion that is injectable and fluid. Semi-solid is generally a rather thicker emulsion or gel which can also be injected if it is not too thick but which has more body and substance than the liquid form. Semi-solid forms might also be pastes or near-solid gels or plugs in addition to emulsions. The solid form is generally a sheet of extracellular matrix. Another solid form is dry particulate which is formed from lyophilized sheets of extracellular matrix and broken up in to fine powder or particulate. Particulate can be reconstituted in a suitable buffer such as saline to become the liquid or semi-solid (gel, emulsion or paste) forms of the extracellular matrix. Particulate can also be used on its own in a dry composition that is placed between sheets of extracellular matrix to form the article. Solid forms can also include strips or other shapes of sheet matrix, or manipulations of the powder form, for example compressed balls of the dry powder, or a sheet rolled up like a manuscript scroll. The sheets in the article can have any number of shapes, e.g. square, rectangular, triangular, circular, or irregular shape. The shape of the article can be tailored to fit the site where the article will be introduced into the body. Accordingly, the compositions that make up the center or encased portion of the article can be any of these forms, encased in one or more sheets of extracellular matrix.

Extracellular matrix can be obtained from the tissues of mammals by processes such as described in U.S. Pat. No. 5,554,389, U.S. Pat. No. 4,902,508, and U.S. Pat. No. 5,281,422. For example, the urinary bladder submucosa is an extracellular matrix that has the tunica mucosa (which includes the transitional epithelial layer and the tunica propria), a submucosal layer, 3 layers of muscularis, and the adventitia (a loose connective tissue layer). This general configuration is true also for small intestine submucosa (SIS) and stomach submucosa (SS). Obtaining enamel matrices is described in U.S. Pat. No. 7,033,611. Enamel matrix is extracellular matrix existing near forming teeth.

Other tissues such as the liver and pancreas have a basement membrane that does not demonstrate the kind of tensile strength of the tissues defined as submucosa. However, other useful properties may be opportunistically employed from the extracellular matrices of such tissues as the liver, pancreas, placenta and lung tissues which have either basement membrane for extracellular matrix or interstitial membrane (as with the lung). These softer matrices support cells such as those in the organs from which the matrices are derived. Thus, certain benefits are to be found in using the extracellular matrices of these tissues, especially in combination with other such matrices like SIS and SS that may be stronger and which offer their particular advantages. The extracellular matrices surrounding developing tooth enamel and developing bone also have particular advantages over other matrices in that they support the growth and differentiation of the hard tissues of bone and enamel. Accordingly, the liver, lung, and pancreatic extracellular matrices may be quite suitable for generating a powder form of extracellular matrix, and from that an emulsion form by rehydrating the powder, and then this emulsion can be placed in between two sheets of extracellular matrix with tensile strength such as SIS or SS, and form an article that can promote healing of a wound or regeneration of tissue at a defect in the mammal. For some sites in a human being treated, such a combination of matrices in the article described may work to optimally heal the wound or regenerate the tissue.

Matrices can be used in whole or in part, so that for example, an extracellular matrix can contain just the basement membrane (or transitional epithelial layer) with the sub-adjacent tunica propria, the tunica submucosa, tunica muscularis, and tunica serosa. The matrix composition can contain any or all of these layers, and thus could conceivably contain only the basement membrane portion, excluding the submucosa. However, generally, and especially since the submucosa is thought to contain and support the active growth factors and other proteins necessary for in vivo tissue regeneration, the matrix composition from any given source will contain the active extracellular matrix portions that support cell development and differentiation and tissue regeneration once placed in a live mammal. Thus it is generally understood by persons of skill in the art that the extracellular matrix of any of the mammalian tissue consists of several basically inseparable layers broadly termed extracellular matrix. Where layers can be separated these separate layers can electively be included in the composition, depending on whether they serve the purpose that is the goal of the article.

Extracellular matrix can be made into a particulate and fluidized as described in U.S. Pat. No. 5,275,826 to Badylak, U.S. Pat. No. 6,579,538 to Spievack, and U.S. Pat. No. 6,933,326 to Griffey. Fluidized or emulsified compositions (the liquid or semi-solid forms) can be present at a certain concentration, for example at a concentration of extracellular matrix greater than about 0.001 mg/ml. The concentration of these liquid or semi-solid components of the extracellular matrix composition can be in a range from about 0.001 mg/mi to about 200 mg/ml. The concentrations can further be found in more specific ranges such as for example the following set of ranges: about 5 mg/mi to about 150 mg/ml, about 10 mg/ml to about 125 mg/ml, about 25 mg/ml to about 100 mg/ml, about 20 mg/ml to about 75 mg/ml, about 25 mg/ml to about 60 mg/ml, about 30 mg/ml to about 50 mg/mi, and about 35 mg/ml to about 45 mg/mi, and about 40 mg/ml. to about 42 mg/ml. This set of ranges is exemplary and not intended to be exhaustive, it is contemplated that any value within any of these specifically listed ranges is a reasonable and useful value for a concentration of a liquid or semi-solid component of the composition.

The composition that is encased by one or more sheets of extracellular matrix can comprise extracellular matrix combinations from such sources as, for example but not limited to, small intestine submucosa, liver basement membrane, stomach submucosa, urinary bladder submucosa, placental basement membrane, pancreatic basement membrane, large intestine submucosa, lung interstitial membrane, respiratory tract submucosa, heart extracellular matrix, dermal matrix, and in general extracellular matrix from any mammalian fetal tissue. Any one of these tissue sources can provide extracellular matrix that can then be manipulated into a designated form (liquid, semisolid, or solid form), for use in a composition.

The compositions of the invention that are encased by the sheets of extracellular matrix can be made from a single source of extracellular matrix. The composition can also be made from two or more extracellular matrices isolated from a donor mammal or from a particular tissue source in that donor or multiple donors. So that the composition is made up of emulsion or particulate of an extracellular matrix from one tissue source from a mammal, and also emulsion or particulate from another extracellular matrix from a different tissue source from either the same mammal, the same species of mammal, or from an entirely different mammalian animal or an entirely different species of mammal. In any event, the key factor is that at least two tissue sources from which the composition comprising mammalian extracellular matrix can be derived to form the composition derived from different tissue sources.

The composition can be made from three mammalian tissue sources, four mammalian tissue sources, five mammalian tissue sources, six mammalian tissue sources, and conceivably up to ten or more tissue sources. Once again these tissue sources can be from the same mammal (for example the same cow, the same pig, the same rodent, the same human, etc.), the same species of mammal (e.g. a cow, a pig, a rodent, a human), or different mammalian animals (but the same species, e.g. cow 1 and cow 2, or pig 1 and pig 2), or different species of mammals (for example liver matrix from a pig, and small intestine submucosa from a cow, and urinary bladder submucosa from a dog).

Accordingly, the composition can be made entirely for example of small intestine submucosa (SIS), but some of the matrix can be in laminate sheets, and some of the matrix can be in an emulsion or semi-solid form that is placed within two laminate sheets (like a sandwich). The ends of the sheets can be sealed to each other, encasing a gel, emulsion, paste, liquid or particulate form of SIS within sheets of SIS.

The matrices will generally be in a liquid form, a semi-solid (emulsion, gel or paste) form, or a solid form (generally a solid sheet or strip or a dry particulate). Each physical form of extracellular matrix has its particular advantages and can be used to engineer the final composition of any of the extracellular matrix compositions of the invention. For example, the liquid forms can be present in a range of concentrations, from very dilute at about 0.001 mg/ml to greater concentrations of up to about 200 mg/ml. The emulsion will be more concentrated than the liquid form and will retain a shape which can be useful in applying the matrix composition to certain parts of the body. The emulsion can be concentrated enough to form shapes like a plug or other configuration suited to the site at which the matrix composition is being applied. Thick emulsion or paste can be painted or otherwise applied at a site, and dusted with solid particulate on top of the emulsion. Solid forms of the extracellular matrix can include sheets, particulate or powder, strips, sheets cut in particularly useful shapes, strands that can be used to apply stitches at a site, and other solid forms. The solid particulate can be reconstituted to form the emulsion, or can be applied dry as a particulate powder which can dust a region in the subject being treated, or be encased in sheets or pockets of extracellular matrix.

Where the composition comprises dry particulate, the dry particulate of two matrices can be mixed together in some proportion. For example, 50% of particulate small intestine submucosa can be mixed with 50% of particulate pancreatic basement membrane in a vial. Where the composition comprises an emulsion, a dry particulate mixture such as this can then be fluidized by hydrating it in a suitable buffer, for example saline. The same is true for a gel, paste, or liquid extracellular matrix forming the composition filler.

The hydration of any particulate (from a single source of extracellular matrix or from a mixture as just described) can be accomplished to a desired concentration of extracellular matrix, for example in a range from about 0.001 mg/ml to about 200 mg/ml. The concentrations can further be found in more specific ranges such as for example the following set of ranges: about 5 mg/ml to about 150 mg/ml, about 10 mg/ml to about 125 mg/ml, about 25 mg/ml to about 100 mg/ml, about 20 mg/ml to about 75 mg/ml, about 25 mg/ml to about 60 mg/ml, about 30 mg/ml to about 50 mg/ml, and about 35 mg/ml to about 45 mg/ml, and about 40 mg/ml. to about 42 mg/mi. This set of ranges is exemplary and not intended to be exhaustive. It is contemplated that any value within any of these specifically listed ranges is a reasonable and useful value for a concentration of a liquid or semi-solid of the composition.

The lower the concentration of extracellular matrix the more liquid the composition will be and the higher the concentration of extracellular matrix the more the composition approaches a gel-like or semi-solid consistency.

For the composition that is encased by the sheet or sheets of extracellular matrix, if the composition is a mixture of extracellular matrixes from different tissues, the ratio of the mixtures of the two liquid or semi-solid extracellular matrices from different sources (or the same source) can be unequal. So for example, LBM can be present at 75% and SIS can be present at 25%. Any suitable ratio can be used: 1:1, 1:2, 1:3, 1:4, 1:5, and so on. Where 3 or more tissue sources of ECM are represented in the composition, the same type of balance or inbalance in the amounts of the matrices can occur. For ECM from 3 sources, each source can be present in a third, 1:1:1, or a disproportionate amount of the particulate can be from one source, say 50% and the other two sources can be present in equal or unequal amounts (relative to each other), so as 25% each, or one can be present as 30% and the other as 20%, so 1:2:3, and 1:1:2 ratios for example. In such compositions the two or more matrices can be fluidized or emulsified together or separately. Rehydration of the dry particulate acellular extracellular matrix to form a liquid or emulsion can be accomplished just prior to use in the composition. These ratios are intended to be exemplary and not exhaustive of the possible ratios that will work in designing a composition filler for the article.

The composition comprising mammalian extracellular matrix in a gel, emulsion, paste, liquid or particulate form can further include one or more additional components to aid in some aspect of the tissue regenerative process. The additional component will generally be part of the composition that it used on its own, or that composition that is used as a filler that is placed between the sheets of matrix shaped to conform to the human breast or other part of the human anatomy. Thus, the additional component can help to regenerate tissue, heal a wound, better recruit stem cells, manipulate the immune environment in a beneficial way, augment tissue, therapeutically treat the local environment, or otherwise contribute to some aspect of the process for which the composition is being used.

Thus, the additional component can be a cell, a protein or a drug. The cell can be a stem cell, such as, for example a of human embryonic stem cell, a fetal cardiomyocyte, a myofibroblast, a mesenchymal stem cell, an autotransplanted expanded cardiomyocyte, an adipocyte, a totipotent cell, a pluripotent cell, a blood stem cell, a myoblast, an adult stem cell, a bone marrow cell, a mesenchymal cell, an embryonic stem cell, a parenchymal cell, an epithelial cell, an endothelial cell, a mesothelial cell, a fibroblast, a myofibroblast, an osteoblast, a chondrocyte, an exogenous cell, an endogenous cell, a stem cell, a hematopoetic stem cell, a pluripotent stem cell, a bone marrow-derived progenitor cell, a progenitor cell, a myocardial cell, a skeletal cell, a fetal cell, an embryonic cell, an undifferentiated cell, a multi-potent progenitor cell, a unipotent progenitor cell, a monocyte, a cardiomyocyte, a cardiac myoblast, a skeletal myoblast, a macrophage, a capillary endothelial cell, a xenogenic cell, an allogenic cell, an adult stem cell, and a post-natal stem cell. This list is not intended to be exhaustive.

The protein can be for example a growth factor, or any other type or protein that might stimulate some part of the tissue regenerative process, a collagen, a proteoglycan, a glycosaminoglycan (GAG) chain, a glycoprotein, a growth factor, a cytokine, a cell-surface associated protein, a cell adhesion molecule (CAM), an angiogenic growth factor, an endothelial ligand, matrikine, a matrix metalloprotease, a cadherin, an immunoglobin, a fibril collagen, a non-fibrillar collagen, a basement membrane collagen, a multiplexin, a small-leucine rich proteoglycan, decorin, biglycan, a fibromodulin, keratocan, lumican, epiphycan, a heparan sulfate proteoglycan, periecan, agrin, testican, syndecan, glypican, serglycin, selectin, a lectican, aggrecan, versican, nuerocan, brevican, cytoplasmic domain-44 (CD-44), macrophage stimulating factor, amyloid precursor protein, heparin, chondroitin sulfate B (dermatan sulfate), chondroitin sulfate A, heparan sulfate, hyaluronic acid, fibronectin (Fn), tenascin, elastin, fibrillin, laminin, nidogen/entactin, fibulin I, fibulin II, integrin, a transmembrane molecule, platelet derived growth factor (PDGF), epidermal growth factor (EGF), transforming growth factor alpha (TGF-alpha), transforming growth factor beta (TGF-beta), fibroblast growth factor-2 (FGF-2) (also called basic fibroblast growth factor (bFGF), thrombospondin, osteopontin, angiotensin converting enzyme (ACE), and vascular epithelial growth factor (VEGF). This list is not intended to be exhaustive.

The additional component can also be a drug, such as an agent that has therapeutic properties. The drug can be bioactive and play some role in the process of tissue regeneration, for example, or act as an antibiotic, antiviral, or other active therapeutic agent serving a purpose in the composition as a whole, also by example. The drug can be a small molecule, or any other agent having therapeutic properties.

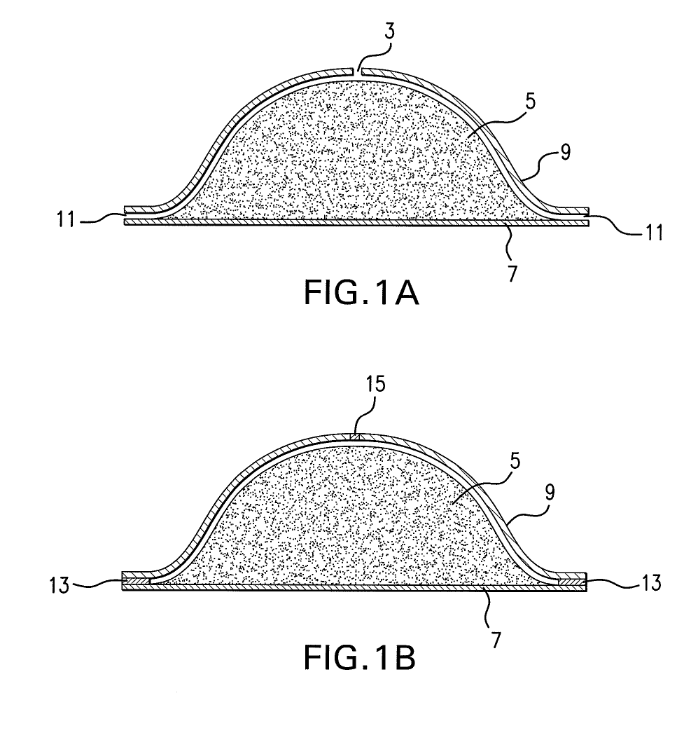

Turning now to the Figures, FIG. 1A depicts a cross sectional view of a breast implant article having at least one sheet of extracellular matrix, 9, and another sheet 7 encasing a composition of extracellular matrix, 5. Openings 3 and 11 indicate that the sheets of extracellular matrix have not been laminated together in this drawing. FIG. 1B depicts the same cross sectional drawing with openings 13 and 15 laminated closed. Sheet 9 and 7 encase the composition 5 as in FIG. 1A. The article can be made using bottom sheet 7 cut in a circular or elliptical shape to conform to the base of a human breast. 2 or 4 sheets shaped like sheet 9, to contour and raise up the article to create a space for placing a space-filling composition 5 into the article can then be placed on top of and laminated to base sheet 7. Lamination can take place at the edges of sheet 7, and where the sheets shaped like sheet 9 meet to form the top of the breast-shaped article. So that space 3 is closed upon lamination of sheet 9 to other neighboring sheets of similar shape to form opening 15 shown in FIG. 1B as a closed and laminated area. Space 11 is closed upon lamination to form closed seal 13 in FIG. 1B. Within the article is placed (before closure or lamination) composition 5 which is an extracellular matrix composition. This composition should fill or nearly fill the space created by the base sheet 7 and the sheets shaped like sheet 9 that form the contoured breast-shaped article. Upon closure or lamination of the sheets together, the composition 5 is encased in the article and ready for placement in the human breast.

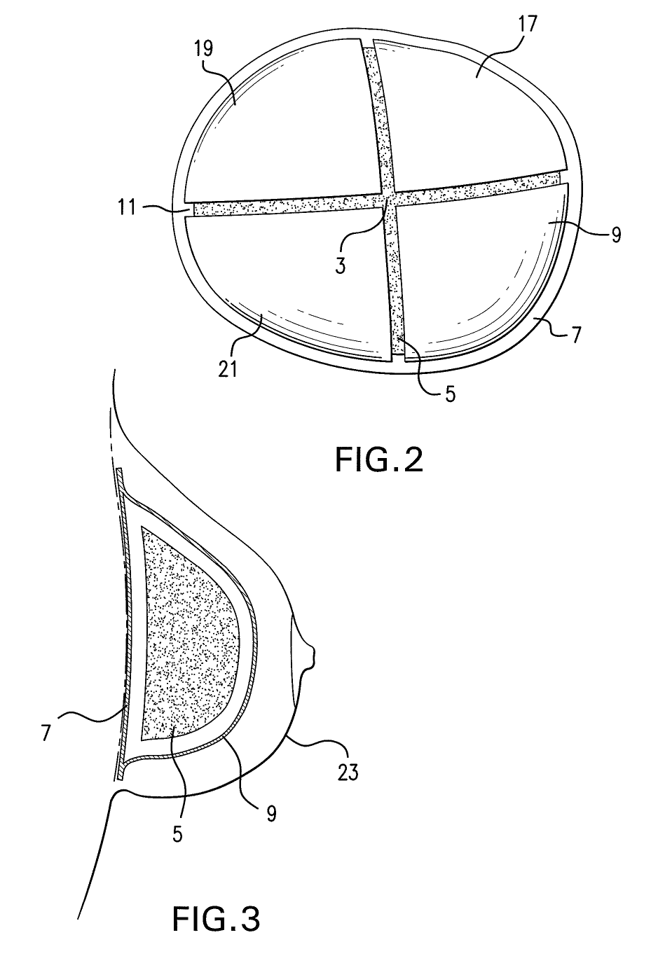

FIG. 2 depicts a top view of an implant having bottom sheet 7, and top sheets 9, 17, 19, and 21. Opening 3 shows that the sheets have not been laminated together. Composition 5 is visible through opening 4. Opening 11 indicates that the sheets have not been laminated to bottom sheet 7 or the top sheets 17, 19 and 21. Here top sheets 9, 17, 19 and 21 depict one pattern of top sheets possible in forming the article. Other patterns and shapes of sheets are possible to construct the three-dimensional contoured breast-shaped article, for example 2 rather than 4 sheets, or one sheet, where it is possible to make and contour such a large piece of extracellular matrix. Sheet 7, the bottom sheet of the article, might also of necessity be made from more than one sheet of extracellular matrix that is connected with other sheets to form the circular base depicted in FIG. 2. In FIG. 2 composition 5 is visible through the unclosed space of 3, but after closure the composition is no longer visible, provided the closure is complete.

FIG. 3 depicts a cross sectional view of an implant in a human breast 23 having top sheet 9, and bottom sheet 7, and composition 5. Incisions can be made in the areola to introduce the implant, or as needed in the breast and as assessed by the surgeon performing the operation. The implant can be placed in the breast as depicted in FIG. 3, with the base of the implant 7, seated against the wall of the breast cavity and the contoured portion of the implant, 9, conforming to the basic shape of the human breast. The composition of extracellular matrix 5 is encased in the article. As the implant is biodegraded and incorporated into the human breast, the shape of the implant and the generation of new tissue that will ensue may alter the original shape of the implant to conform more completely to the natural shape of the breast into which the implant is introduced. For the purposes of augmentation of the breast, the additional tissue provided by the implant as new tissue is generated due to the presence of the implant in the breast will account for the increased size of the breast.

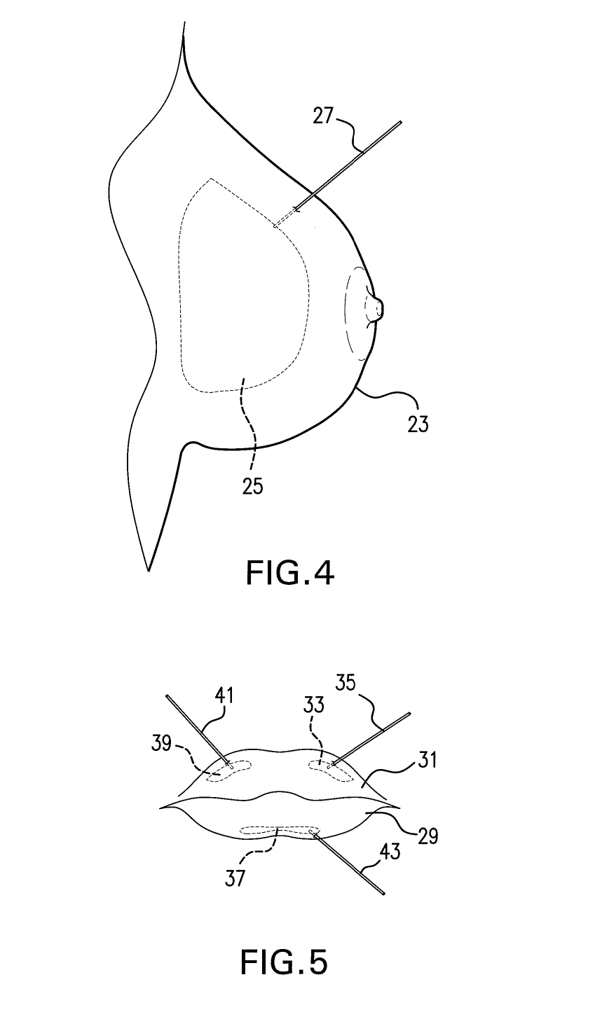

FIG. 4 depicts a cross sectional view of a composition of extracellular matrix being injected using tool 27 into breast 23 to create pocket 25 of extracellular matrix composition. Tool 27 can be a cannula, or catheter, or other injection device capable of injecting a composition of extracellular matrix. The viscosity of the composition may dictate what type of device and how wide the lumen of such device needs to be. For example, liquid extracellular matrix may be injected with a fine lumen, and viscous extracellular matrix (such as a thick emulsion or gel) will need a larger lumen. Preferably the volume of the composition is predetermined based on the desired size of the breast. So that for example, a volume in the range from about 180 cc to about 300 cc will be generally an acceptable range for augmentation purposes. For reconstructive purposes, the surgeon will assess the volume necessary to accomplish the reconstructive goals of the procedure. If possible, the achieved volume can be assessed during the procedure and more composition can be introduced if needed.

FIG. 5 depicts a front-on view of bottom lip 31 having tool 35 inject an aliquot of the extracellular matrix 33 into top lip 31. Likewise top lip 31 has another aliquot of extracellular matrix 39 injected with tool 41, and bottom lip 29 having a third aliquot of matrix 37 injected using tool 43. Injection of the composition 33, 39, and 37 can be done with a fine, or relatively fine lumen device in order to minimize trauma to the lips from the procedure. Most efficiently a predetermined volume of extracellular matrix composition is introduced in the positions identified in the figure, although each patient might have subtle variations in the locations and amount of material introduced depending on the aesthetic goals of the procedure.

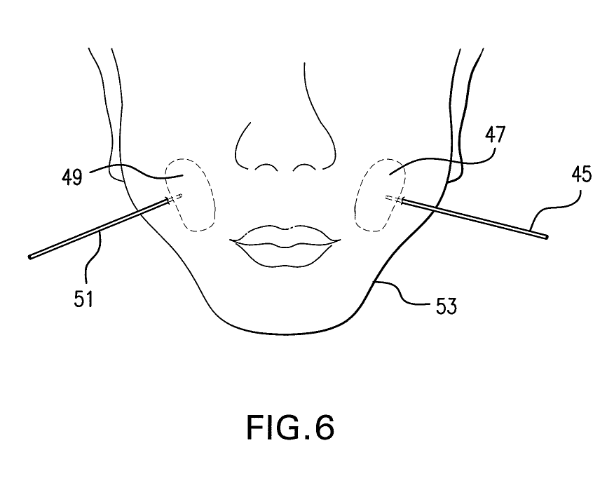

FIG. 6 depicts extracellular matrix being injected into the human face in two aliquots 47 and 49 using tools 45 and 51 respectfully. As any region of the body can be augmented using the extracellular matrix composition, such locations as the cheeks and lips (FIG. 5) are exemplary. Shown here in FIG. 6, sallow cheeks can be enhanced with extracellular matrix composition introduced using device 45 and device 51 to introduce composition 47 and 49 in their respective right and left cheek positions in face 53. The exact volume of material introduced and the position of introduction on the face 53 is determined by the surgeon performing the procedure in conjunction with patient input before surgery. Other locations of the body where such a composition might be used to enhance the contours of the body and appearance might include the buttocks and legs or in general any part of the body that a particular patient desires to be augmented or otherwise receive a shape revision by virtue of introduction of the extracellular matrix composition.

The invention contemplates using the articles of extracellular matrices for contacting a defect in mammalian tissue or an aberration in shape. The defect or aberration can be a cut, disease, wound, burn, scar, necrosis, or other abnormality that would be beneficial to the patient to treat. The defect can also be congenital, or otherwise having been developed by virtue of aging. Regenerating tissue at the defect can be one response elicited from the step of placing the extracellular matrix composition in contact with the defect. Augmentation of tissue can be desired for aesthetic purposes. If the defect is a wound in need of healing, such as after reconstructive surgery, wound healing may be another response that occurs as a result of placing the extracellular matrix at the wound site. In general any term that identifies that the tissue could benefit from healing or where the concept of tissue regeneration fits within the scope of the use for the composition, or the concept of augmentation or aesthetic remodeling, can be used to describe the process that is the goal of placing the article in the patient. Thus regenerating tissue, or healing a wound, augmenting tissue, or aesthetic remodeling are but a few but not the only phrases that can be used to describe the effects achieved when the composition is placed in the mammal at a site of wound, defect or damage in tissue.

Therapeutically effective amount is a term meant to capture the idea that you need to apply enough of the composition that is used within a laminate article or enough of the composition in sufficient strength where the composition is placed in the body by itself so that the composition can have a positive effect on the tissue that is being treated in the subject. The positive effect can comprise tissue augmentation, or tissue regeneration, wound healing and the like. The amount may therefore apply to a quantity of matrix, or a size of a sheet of matrix, or a volume or weight of powder, or a concentration of liquid, gel or emulsion. That the amount is therapeutically effective is determined by the composition's ability to have a regenerative or wound healing effect or tissue augmentation effect at the site where the composition contacts the tissue. A therapeutically effective amount is determinable by routine testing in patients with wounds or defects. In general a minimal therapeutically effective amount would be considered sufficient composition to contact amply all of the wound or defect in the tissue.

Regenerating tissue is the ability to make tissue regrow, an organ regrow itself, and for new tissue to reform without scarring. Healing a wound is the ability of the tissue to heal without scarring, or with less scarring than would have occurred without the article. Augmenting tissue is providing new material from which new tissue can form within the body.

All references cited are incorporated in their entirety. Although the foregoing invention has been described in detail for purposes of clarity of understanding, it will be obvious that certain modifications may be practiced within the scope of the appended claims.

What is claimed is:

1. An article for placing in a human breast comprising a composition comprising extracellular matrix in an emulsion, gel, paste, liquid or particulate form, wherein the article forms an implant shaped to conform to a shape of a human breast.

2. The article of claim 1, wherein said encased composition comprises a concentration of extracellular matrix greater than about 0.001 mg/ml.

3. The article of claim 1, wherein said encased composition comprises a concentration of extracellular matrix in a range from about 0.001 mg/ml to about 200 mg/ml.

4. The article of claim 1, wherein said encased composition comprises extracellular matrices from two or more mammalian tissue sources.

5. The article of claim 1, wherein said sheets of extracellular matrix comprise small intestine submucosa, or stomach submucosa.

6. The article of claim 1, wherein said encased composition further comprises an additional component.

7. The article of claim 6, wherein said additional component is a cell.

8. The article of claim 7, wherein said cell is a stem cell.

9. The article of claim 6, wherein said additional component is a protein.

10. The article of claim 9, wherein said protein is a growth factor.

11. The article of claim 6, wherein said additional component is a drug.

12. A method of augmenting or reconstructing a human breast comprising:
a) providing an article comprising one or more sheets of mammalian extracellular matrix encasing a composition comprising extracellular matrix in an emulsion, gel, paste, liquid or particulate form, wherein said article forms an implant shaped to conform to a shape of a human breast, and
b) placing said article in a human breast.

13. The method of claim 12, wherein said sheets comprise small intestine submucosa or stomach submucosa.

14. The method of claim 12, wherein said encased composition comprises extracellular matrices from two or more mammalian tissue sources.

15. The method of claim 12, wherein said encased composition further comprises an additional component.

16. The method of claim 15, wherein said additional component is a cell.

17. A method of augmenting tissue at a site in the human body comprising:
a) providing a composition comprising mammalian extracellular matrix, and
b) introducing said composition into said human body in sufficient amount to generate new tissue at said site, resulting in an augmentation of tissue at said site.

18. The method of claim 17, wherein said composition comprises extracellular matrix from more than one mammalian tissue source.

19. The method of claim 17, wherein said composition comprises extracellular matrix in a concentration greater than about 0.001 mg/ml.

20. The method of claim 17, wherein said site in said human body is a breast.
